(19) United States
(12) Patent Application Publication
(10) Pub. No.: US 2005/0003418 A1
(43) Pub. Date: Jan. 6, 2005

(54) Multiplex PCR primer set for amplifying human MODY genes 1,4,5,6 and 7

(76) Inventors: Mi-kyung Kim, Daejeon-si (KR); Hyo-jeong Han, Gyeonggi-do (KR); Soo-jung Kim, Gyeonggi-do (KR); Sung-young Jeong, Gyeonggi-do (KR); Kui-hyun Kim, Daejeon-si (KR); Jung-nam Lee, Daejeon-si (KR); Yoon-jung Choi, Chungcheongnam-do (KR)

Correspondence Address: CANTOR COLBURN, LLP, 55 GRIFFIN ROAD SOUTH, BLOOMFIELD, CT, 06002, US

(21) Appl. No.: 10/871,302

(22) Filed: Jun. 19, 2004

(30) Foreign Application Priority Data
Jun. 17, 2003 (KR) 2003-39125

(51) Int. Cl. C12Q 1/68; C07H 21/04; C12P 19/34

(52) U.S. Cl. 435/6; 435/91.2; 536/24.3

(57) ABSTRACT

A primer pool including at least two sets of primers for amplifying at least two target sequences of human MODY gene 1, 4, 5, 6, or 7, the at least two sets of primers being selected from the group consisting of sets of primers, each set including an oligonucleotide having one of SEQ ID NOS. 1 through 32, 41 and 42 and its variant oligonucleotide.

### BACKGROUND OF THE INVENTION

This application claims the priority of Korean Patent Application No. 2003-39125 filed on Jun. 17, 2003, in the Korean Intellectual Property Office, the disclosure of which is incorporated herein in its entirety by reference.

1. Field of the Invention

The present invention relates to a primer pool for multiplex PCR, a method of analyzing a nucleotide sequence using the primer pool, and a kit for amplifying the target sequence using the primer pool.

2. Description of the Related Art

A method of detecting hybridized nucleotides using a polymerization chain reaction is widely known in the field (U.S. Pat. Nos. 4,683,195; 4,683,202; and 4,800,159). The PCR reaction is achieved by repeated cycles of denaturation, annealing for hybridizing a target sequence of a sample with a complementary primer, and polymerization using a thermally stable DNA polymerase to extend a DNA double helix from the hybridized primer. If no nucleotide primer hybridizes to the target nucleic acid, there is no PCR product. The PCR primer acts as a hybridization probe.

Regarding the PCR method, the amplified PCR products can be identified using various techniques, for example, by inserting a labeled nucleotide into the strands amplified using labeled primers. Examples of labeling materials include, but are not limited to, radioactive materials, fluorescent dyes, digoxygenin, horseradish peroxidase, alkaline phosphatases, acridium ester, biotin, and jack beam urease. Furthermore, the PCR products obtained using non-labeled primers can be identified by combination of gel separation using electrophoresis and a dye-based visualizing technique.

The Human genome is composed of about 3×10⁹ nucleotides, and thus it is a difficult to isolate and analyze a specific human gene. PCR technologies have been developed to amplify a specific sequence. A PCR can amplify a target sequence with a high speed, specificity and sensitivity by using a set of primers including primers complementary to both ends of the target sequence.

PCR is widely used in analyzing a disease-associated gene. Specifically, gene amplification by PCR is useful for analyzing genetic variations of a disease-associated gene in the medical field. A specific disease-associated gene is amplified using PCR, and analyzed by using a sequencing, hybridization or single strand conformational polymorphism. Where a genetic variation of a gene is mentioned herein, it means a change in a nucleotide sequence including a deletion, addition or inversion of a nucleotide sequence. Specifically, a genetic variation of a gene includes a single nucleotide polymorphism.

In the analysis of genetic variations of a gene, if the size of a target gene is small, a single PCR may be enough to amplify the entire gene. However, if the size of a target gene is large, for example, 1 kb or more, a single PCR may not be able to amplify the entire gene. Thus, the PCR should be separately conducted several times on several portions of the entire gene to amplify the entire gene. In the analysis of a genetic variation of a disease-associated gene, a multiple PCR is more frequently used than a single PCR since most disease-associated genes have a size of 1.5 kb or more.

However, a multiple PCR requires a large amount of a sample, for example, a patient's DNA or blood. A multiple PCR also costs more and requires more effort and time.

A multiplex PCR has been developed to solve the above problems. A multiplex PCR simultaneously amplifies a plurality of target sequences of a gene in one tube reaction. Therefore, a plurality of target sequences are amplified by a single PCR using a primer pool for amplifying each target sequence.

A multiplex PCR using such a primer pool, a set of primers can save time, effort and cost for amplifying a target sequence in comparison with a single PCR. Specifically, in the analysis of a genetic variation of a gene by using a DNA chip, a multiplex PCR is useful in amplifying more than one kind of DNA sample in a reaction.

It is known that genetic variations in MODY (maturity-onset diabetes mellitus in the young) gene can cause MODY. It is estimated that MODY accounts for about 10-30% of Type II MODY (Matschinsky & Magnuson, in ‘Molecular Pathogenesis of MODYs’, Karger, 16-33, 2000). Thus, by analyzing variations in MODY genes, it is possible to anticipate a person's propensity to the diabetes mellitus. Therefore, in order to rapidly analyze generic variations, for example, in MODY genes using DNA chips, it is needed to develop a set of primers for amplifying human MODY genes.

### SUMMARY OF THE INVENTION

The present invention provides a primer pool including sets of primers for amplifying a target sequence of human maturity onset diabetes mellitus (MODY) gene 1, 4, 5, 6, or 7 by a multiplex polymerization chain reaction (PCR).

The present invention also provides a method of amplifying a target sequence of human MODY gene 1, 4, 5, 6, or 7 using the primer pool.

The present invention also provides a method of analyzing a target nucleotide sequence of human MODY gene 1, 4, 5, 6, or 7 using the primer pool.

The present invention also provides a kit for amplifying a target sequence and including the primer pool.

According to an aspect of the present invention, there is provided a primer pool including at least two sets of primers for amplifying at least two target sequences of human MODY gene 1, 4, 5, 6, or 7, the at least two sets of primers being selected from the group consisting of:
(a) a set of primers including an oligonucleotide having SEQ ID NO. 1 or its variant oligonucleotide and an oligonucloetide having SEQ ID NO. 2 or its variant oligonucleotide;
(b) a set of primers including an oligonucleotide having SEQ ID NO. 3 or its variant oligonucleotide and an oligonucloetide having SEQ ID NO. 4 or its variant oligonucleotide;
(c) a set of primers including an oligonucleotide having SEQ ID NO. 5 or its variant oligonucleotide and an oligonucloetide having SEQ ID NO. 6 or its variant oligonucleotide;
(d) a set of primers including an oligonucleotide having SEQ ID NO. 7 or its variant oligonucleotide and an oligonucloetide having SEQ ID NO. 8 or its variant oligonucleotide;
(e) a set of primers including an oligonucleotide having SEQ ID NO. 9 or its variant oligonucleotide and an oligonucloetide having SEQ ID NO. 10 or its variant oligonucleotide;
(f) a set of primers including an oligonucleotide having SEQ ID NO. 11 or its variant oligonucleotide and an oligonucloetide having SEQ ID NO. 12 or its variant oligonucleotide;
(g) a set of primers including an oligonucleotide having SEQ ID NO. 13 or its variant oligonucleotide and an oligonucloetide having SEQ ID NO. 14 or its variant oligonucleotide;
(h) a set of primers including an oligonucleotide having SEQ ID NO. 15 or its variant oligonucleotide and an oligonucloetide having SEQ ID NO. 16 or its variant oligonucleotide;
(i) a set of primers including an oligonucleotide having SEQ ID NO. 17 or its variant oligonucleotide and an oligonucloetide having SEQ ID NO. 18 or its variant oligonucleotide;
(j) a set of primers including an oligonucleotide having SEQ ID NO. 19 or its variant oligonucleotide and an oligonucloetide having SEQ ID NO. 20 or its variant oligonucleotide;
(k) a set of primers including an oligonucleotide having SEQ ID NO. 21 or its variant oligonucleotide and an oligonucloetide having SEQ ID NO. 22 or its variant oligonucleotide;
(l) a set of primers including an oligonucleotide having SEQ ID NO. 23 or its variant oligonucleotide and an oligonucloetide having SEQ ID NO. 24 or its variant oligonucleotide;
(m) a set of primers including an oligonucleotide having SEQ ID NO. 25 or its variant oligonucleotide and an oligonucloetide having SEQ ID NO. 26 or its variant oligonucleotide;
(n) a set of primers including an oligonucleotide having SEQ ID NO. 27 or its variant oligonucleotide and an oligonucloetide having SEQ ID NO. 28 or its variant oligonucleotide;
(o) a set of primers including an oligonucleotide having SEQ ID NO. 29 or its variant oligonucleotide and an oligonucloetide having SEQ ID NO. 30 or its variant oligonucleotide;
(p) a set of primers including an oligonucleotide having SEQ ID NO. 31 or its variant oligonucleotide and an oligonucloetide having SEQ ID NO. 32 or its variant oligonucleotide;
(q) a set of primers including an oligonucleotide having SEQ ID NO. 41 or its variant oligonucleotide and an oligonucloetide having SEQ ID NO. 16 or its variant oligonucleotide;
(r) a set of primers including an oligonucleotide having SEQ ID NO. 42 or its variant oligonucleotide and an oligonucloetide having SEQ ID NO. 18 or its variant oligonucleotide;
wherein said variant oligonucleotide is an oligonucleotide having 1 to 3 additional nucleotides joined or deleted from the 3′ end, the 5′ end, or both the 3′ end and the 5′ end of the corresponding oligonucleotide. The 1 to 3 additional nucleotides joined to the corresponding oligonucleotide are preferably complementary to the target nucleic acid.

According to another aspect of the present invention, there is provided a method of amplifying at least two target sequences of human MODY gene 1, 4, 5, 6, or 7, the method including performing a polymerization chain reaction (PCR) on the at least two target sequences using the primer pool.

According to another aspect of the present invention, there is provided a method of analyzing at least two target nucleic acids of human MODY gene 1, 4, 5, 6, or 7 using the primer pool. In the analyzing method, the primer pool is used as sequencing primers for the amplified target sequence. The sequencing method may include general sequencing processes using the primer pool as sequencing primer.

According to another aspect of the present invention, there is provided a kit for amplifying a target sequence of human MODY gene 1, 4, 5, 6, or 7 and including the primer pool of the present invention. The kit may further include general PCR reagents, such as a dNTP solution, a DNA polymerase, and a buffer.

A primer pool according to the present invention can be used to detect genetic variations in human MODY gene 1, 4, 5, 6, or 7 that accounts for about 10-30% of Type II diabetes. The genetic propensity of each individual can be anticipated by analyzing variations in human MODY gene 1, 4, 5, 6, or 7. MODY 1 is a type of diabetes caused by a mutation in HNF-4a (Hepatocyte nuclear factor-4a) gene, MODY 4 is a type of diabetes caused by a mutation in insulin promoter factor-1 gene, MODY 5 is a type of diabetes caused by HNF-1 b gene, MODY 6 is a type of diabetes caused by a mutation in neurogenic differentiation factor/beta cell E-box transcription factor 2 (Neuro D1/BETA 2) gene, MODY 7 is a type of diabetes caused by a mutation in islet-1 transcription factor (ISL-1) (J. Mol. Endocrinol. 27:11(2001), J. Clin. Endocrinol. Metab. 86:220 (2001)).

### BRIEF DESCRIPTION OF THE DRAWINGS

The above and other features and advantages of the present invention will become more apparent by describing in detail exemplary embodiments thereof with reference to the attached drawings in which:

FIG. 1 is a photograph illustrating the result of an electrophoresis using the products of a single polymerization chain reaction (PCR) and multiplex PCR on 16 exons of human maturity onset diabetes mellitus (MODY) genes 1, 4, 5, 6, or 7;

FIGS. 2A, 2B, and 2C illustrate the results of sequencing analysis using single PCR and multiplex PCR products of 16 exons of human MODY genes 1, 4, 5, 6, or 7;

FIGS. 3A and 3B are graphs illustrating the results of an analysis of multiplex PCR products of 16 exons of human MODY genes 1, 4, 5, 6, or 7 using a primer pool including 9 sets of primers and a primer pool including 7 sets of primers, respectively, and using a lab chip;

FIGS. 4A and 4B are graphs illustrating the results of an analysis of the products of optimized multiplex PCR on 16 exons of human MODY genes 1, 4, 5, 6, or 7 using a primer pool including 9 sets of primers and a primer pool including 7 sets of primers, respectively, and using a lab chip; and

FIG. 5 is a photograph of the results of an electrophoresis using the products of a multiplex PCR performed using a primer pool including sets of variant primers.

FIG. 6 is a photograph of the results of an electrophoresis using the products of a single PCR and multiplex PCR performed using a primer set and primer pool set forth in Table 5, respectively.

### DETAILED DESCRIPTION OF THE INVENTION

For understanding of the present invention, the definitions of terms used throughout the specification are provided as follows.

The term “nucleic acid” refers to a linear sequence of nucleotides (bases) linked to one another by a phosphodiester bond between 3′-position of a pentose of one nucleotide and 5′-position of a pentose of anther nucleotide. The term “polynucleotide” refers to a nucleic acid including a sequence of nucleotides more than about 100 bases. The term “oligonucleotide” refers to a short polynucleotide or a portion of polynucleotide including about 2-100 bases.

Nucleic acids have been known experiencing various mutations. For example, “point mutation” refers to a mutation in a single base of a nucleotide sequence. “Single nucleotide polymorphism (SNP)” refers to a mutation in the most common base of a nucleotide sequence.

As used herein, the term “target nucleic acid” or “nucleic acid target” refers to a particular nucleic acid sequence of interest. Thus, the “target” can exist in the presence of other nucleic acid molecules or within a larger nucleic acid molecule. In the present invention, target nucleotides include exons of MODY gene 1, 4, 5, 6, or 7.

As used herein, the term “nucleic acid probe” refers to an oligonucleotide or polynucleotide that is capable of hybridizing to another nucleic acid of interest. A nucleic acid probe may occur naturally as in a purified restriction digest or be produced synthetically, recombinantly or by PCR amplification. As used herein, the term “nucleic acid probe” refers to the oligonucleotide or polynucleotide used in a method of the present invention. That same oligonucleotide may also be used, for example, in a PCR method as a primer for polymerization, but as used herein, that oligonucleotide would then be referred to as a “primer”. Herein, oligonucleotides or polynucleotides may contain some modified linkages such as a phosphorothioate bond.

The term “complementary” is used when defining a pair of nucleotide sequences, for example, a base pair of A/T or C/G, that match each other according to the base pairing rules. For example, a sequence of 5′-A-G-T-3′ is complementary to a sequence of 3′-T-C-A-5′. Nucleotide sequences may be “partially” or “perfectly” complementary to one another so that they form partially matching base pairs or perfectly matching base pairs.

The term “homology” refers to a degree to which nucleotide sequences are complementary to one another. Therefore, there may be partial homology or perfect homology between complementary nucleotide sequences.

In developing a primer pool for amplifying a target sequence of human MODY gene 1, 4, 5, 6, or 7 using a multiplex PCR according to the present invention, the following must be considered.

The primer pool should be specific to and able to sufficiently amplify human MODY gene 1, 4, 5, 6, or 7. There should be no interference between primers. Each primer should have a similar Tm value. Each primer should not form a primer pair-dimer, a hairpin, or a primer self-dimer. A microsatellite region and a consecutive-nucleotide region should be excluded.

Hereinafter, the present invention will be described in greater detail with reference to the following examples. The following examples are for illustrative purposes and are not intended to limit the scope of the invention.

Initially, sets of primers for amplifying 16 exons of MODY 1, 4, 5, 6, or 7 (7 from MODY 1, 2 from MODY 4, 5 from MODY 6, 1 from MODY 6, and 1 from MODY 7) was designed. Whether each of the exons could be amplified by single PCR was investigated using the set of primers. It was also investigated using 16 sets of primers, 9 sets of primers, and 7 sets of primers whether each of the exons could be amplified by multiplex PCR. Next, the amplified multiplex PCR products were identified using electrophoresis, a lab chip (Agilant, U.S.A.), and sequencing analysis.

### EXAMPLE 1

### Design of Primers for Amplifying 16 Exons of Human MODY 1, 4, 5, 6, and 7 Genes

Primers were designed such that the size of each PCR product differed by at least 5-10 bp. In designing primers, the following was considered. In particular, the primers should be specific to a target DNA sequence, there should be no interference between the primers, and the primers could sufficiently amplify a target DNA. Each primer should have a similar Tm value, should not form primer pair-dimer, a hairpin, or a primer self-dimer, and should not include four or more identical consecutive nucleotides. A microsatellite region and a consecutive-nucleotide region were excluded from the primer sequences. Interactions between the primers in the designing process were analyzed using a HYBsimulator™ (Advanced Gene Computing Technologies, Inc).

The sequences and characteristics of the designed primers are shown in Table 1 below.

TABLE 1
Primers for amplifying 16 exons of MODY 1, 4, 5, 6, and 7 genes

| | | | | | Size of |
|---|---|---|---|---|---|
| Name of | | | Name of | SEQ ID | PCR Product |
| Gene | Exon No. | Direction | Primer | NO. | (bp) |
| MODY 1 | Exon 2 | F | M1e2f3n | 1 | 534 |
| | | R | M1e2r3n | 2 | |
| | Exon 3 | F | M1e3f2n | 3 | 324 |
| | | R | M1e3r2n | 4 | |
| | Exon 4 | F | M1e4f3n | 5 | 338 |
| | | R | M1e4r3n | 6 | |
| | Exon 7 | F | M1e7f3n | 7 | 459 |
| | | R | M1e7r3n | 8 | |
| | Exon 8 | F | M1e8f3n | 9 | 506 |
| | | R | M1e8r3n | 10 | |
| | Exon 9 | F | M1e9f2n | 11 | 359 |
| | | R | Me19r2n | 12 | |
| | Exon 10 | F | M1e10f2n | 13 | 417 |
| | | R | M1e10r2n | 14 | |
| MODY 4 | Exon 1 | F | M4e1f3n | 15 | 467 |
| | | R | M4e1r2n | 16 | |
| | Exon 2 | F | M4e2f7n | 17 | 267 |
| | | R | M4e2r6n | 18 | |
| MODY 5 | Exon 1 | F | M5e1f2n | 19 | 418 |
| | | R | M5e1r2n | 20 | |
| | Exon 2 | F | M5e2f2n | 21 | 313 |
| | | R | M5e2r2n | 22 | |
| | Exon 3 | F | M5e3f2n | 23 | 230 |
| | | R | M5e3r2n | 24 | |
| | Exon 4 | F | M5e4f2n | 25 | 360 |
| | | R | M5e4r2n | 26 | |
| | Exon 7 | F | M5e7f2n | 27 | 524 |
| | | R | M5e7r2n | 28 | |
| MODY 6 | Exon 2 | F | M6e1f1n | 29 | 588 |
| | | R | M6e1r1n | 30 | |
| MODY 7 | Exon 5 | F | M7e5f2n | 31 | 279 |
| | | R | M7e5r2n | 32 | |

F: forward primer;
R: reverse Primer

### EXAMPLE 2

### Amplification of 16 Exons of Human MODY Genes 1, 4, 5, 6, and 6 Using Single PCR

16 exons of human MODY genes 1, 4, 5, 6, and 7 were amplified by a single PCR using 16 sets of primers prepared in Example 1. The PCR was achieved by initial denaturation (5 min at 95° C.), 30 cycles of denaturartion (30 sec at 95° C.), annealing (15 sec at 64° C.) and polymerization (30 sec at 72° C.), and final extension (3 min at 72° C.). The composition of a reaction solution used in the PCR was as follows:

| | |
|---|---|
| Sterilized DNase-and RNase-free water | 12.8 μl |
| dNTP mix (each nucleotide 2.5 mM) | 2 μl |
| 10 × Taq polymerase buffer | 2 μl |
| a set of primers (each primer 10 pmol) | 2 μl |
| genomic DNA (200-1.0 μg) | 1 μl |
| Taq polymerase (5 unit/μl) | 0.2 μl |

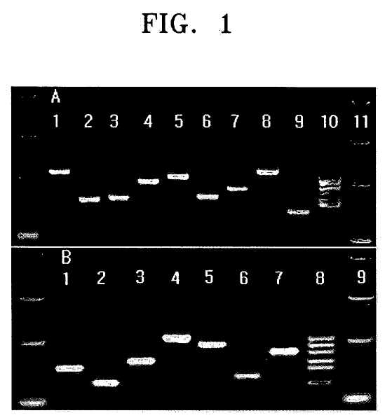

The single PCR products were identified by electrophoresis on 1.8% agarose gel using molecular weight markers. The results are shown in FIG. 1. In group A of FIG. 1, lanes 1 through 6 represent the PCR products for exons 2, 3, 4, 7, 8, and 9 of MODY 1, respectively. Lane 7 represents the PCR products for exon 1 of MODY 5, lane 8 represents the PCR products for exon 2 of MODY 6, lane 9 represents the PCR products for exon 5 of MODY 7, lane 10 represents the PCR products for a primer pool including sets of 9 primers (refer to Example 4), and land 11 represents molecular markers.

In group B of FIG. 1, lanes 1 through 6 represent the PCR products for exons 2, 3, 4, and 7 of MODY 5, respectively. Lanes 5 and 6 represent the PCR products for exons 1 and 2 of MODY 4, respectively. Lane 7 represents the PCR products for exon 5 of MODY 7, lane 8 presents the PCR products for a primer pool including sets of 7 primers (refer to Example 4), and lane 9 represents molecular markers.

It was confirmed from FIG. 1 that the 16 exons of human MODY genes 1, 4, 5, 6, and 7 could be amplified using the set of primers prepared in Example 1.

### EXAMPLE 3

### Amplification of 16 Exons of Human MODY Genes 1, 4, 5, 6, and 7 Using Multiplex PCR

16 exons of human MODY genes 1, 4, 5, 6, and 7 were amplified by a multiplex PCR using 16 sets of primers prepared in Example 1. The PCR was achieved by initial denaturation (5 min at 95° C.), 30 cycles of denaturartion (30 sec at 95° C.), annealing (15 sec at 64° C.) and polymerization (30 sec at 72° C.), and final extension (3 min at 72° C.). The composition of a reaction solution used in the PCR was as follows:

| | |
|---|---|
| Sterilized DNase-and RNase-free water | 14.4 μl or 16.4 μl |
| dNTP mix (each nucleotide 2.5 mM) | 5 μl |
| 10 × Taq polymerase buffer | 5 μl |
| a set of primers (32 primers, 10 pmol each) | 24 μl or 32 μl |
| genomic DNA (200-1.0 μg) | 1 μl |
| Taq polymerase (5 unit/μl) | 0.6 μl |

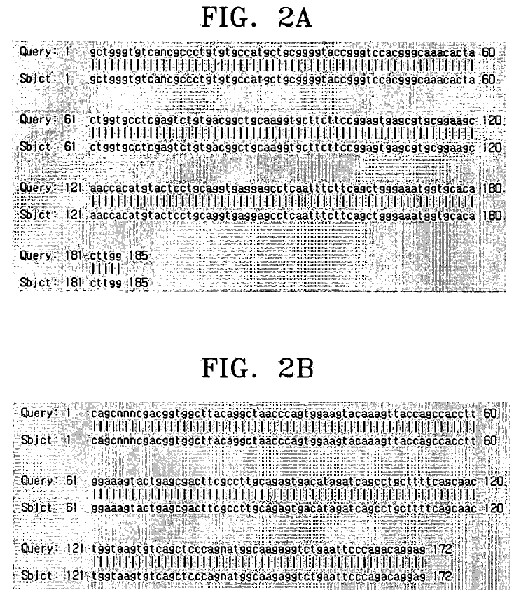

The multiplex PCR products were identified by electrophoresis on 1.8% agarose gel using molecular weight markers. Due to small molecular weight variations in the PCR products, it was difficult to identify whether all the 16 exons could be amplified. Therefore, the multiplex PCR products were purified and analyzed using a general sequencing technique and an ABI 37000. The analyzed sequences of the exons were compared with known consensus sequences using software DNA star™. As a result, the sequences of exon 2 of MODY 1, exon 5 of MODY 7, and exon 1 of MODY 4 showed 100% homology with respect to corresponding consensus sequences, as shown in FIGS. 2A, 2B, and 2C. The other exons showed 98-100% homology with respect to corresponding consensus sequences.

### EXAMPLE 4

### Amplification of 9 Exons and 7 Exons of Human MODY Genes 1, 4, 5, 6, and 7 Using Multiplex PCR

16 sets of primers prepared in Example 1 were grouped into two primer pools, 9 sets of primers and 7 sets of primers. 9 exons and 7 exons of human MODY genes 1, 4, 5, 6, and 7 were separately amplified using the two primer pools. As is apparent from Table 1 above, the primer pool included 9 sets of primers for exons 2, 3, 4, 7, 8, 9 of MODY 1, exon 1 of MODY5, exon 2 of MODY 6, and exon 5 of MODY 7 (group A), and the primer pool included 7 sets of primers for exon 10 of MODY 1, exons 1 and 2 of MODY 4, and exons 2, 3, 4, and 7 of MODY 5 (group B).

The PCR was achieved by initial denaturation (5 min at 95° C.), 30 cycles of denaturartion (30 sec at 95° C.), annealing (15 sec at 64° C.) and polymerization (30 sec at 72° C.), and final extension (3 min at 72° C.). The PCR was performed in a single reaction tube containing the each primer pool, respectively. The compositions of reaction solutions used in the PCR were as follows:

| | |
|---|---|
| Group A | |
| Sterilized DNase-and RNase-free water | 20.4 μl |
| dNTP mix (each nucleotide 2.5 mM) | 5 μl |
| 10 × Taq polymerase buffer | 5 μl |
| a set of primers (18 primers, 10 pmol each) | 18 μl |
| genomic DNA (200-1.0 μg) | 1 μl |
| Taq polymerase (5 unit/μl) | 0.6 μl |
| Group B | |
| Sterilized DNase-and RNase-free water | 24.4 μl |
| dNTP mix (each nucleotide 2.5 mM) | 5 μl |
| 10 × Taq polymerase buffer | 5 μl |
| a set of primers (14 primers, 10 pmol each) | 14 μl |
| genomic DNA (200-1.0 μg) | 1 μl |
| Taq polymerase (5 unit/μl) | 0.6 μl |

The multiplex PCR products were identified by electrophoresis on 1.8% agarose gel (refer to 10 lanes in A of FIGS. 1 and 8 lanes in B of FIG. 1). As shown in FIG. 1, 9 exons (having 88, 534, 506, 459, 418, 359, 338, 324, and 279 bp) and 7 exons (having 524, 467, 417, 360, 313, 265, and 230 bp) of human MODY genes 1, 4, 5, 6, and 7 could be amplified by multiplex PCR using the primer pools. The multiplex PCR products were analyzed using a lab chip (commercially available from Agilant Co., U.S.A.). The lab chip used could perform electrophoresis, identify the size of each PCR product by fluorescently detecting the positions of bands, and calculate the concentration of the PCR product using the height and area of the band.

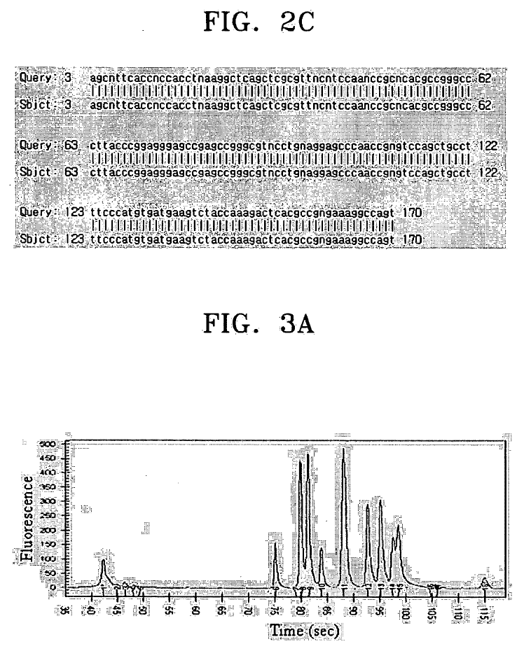

The results of the analysis using the lab chip are shown in FIGS. 3A and 3B. Bands from 9 exons are apparent in FIG. 3A and bands from 7 exons are apparent in FIG. 3B. However, the concentrations of exon 5 of MODY 7 (the first band from the left in FIG. 3A), exon 3 (the fourth band in FIG. 3A) of MODY 1, and exon 0.2 (the second band from the left in FIG. 3B) of MODY 4 were too low and needed to be optimized.

To this end, the concentrations of primers for the exons were varied to the ranges shown in Table 2 below such that each of the exons could be amplified to a concentration of 11 nmol/L or more by multiple PCR.

TABLE 2

| | | | | Concentration |
|---|---|---|---|---|
| Group | Gene | Exon No. | Direction | of Primer (μM) |
| B | MODY1 | Exon 2 | F | 0.1˜0.3 |
| | | | R | 0.1˜0.3 |
| | | Exon 3 | F | 0.05˜0.2 |
| | | | R | 0.05˜0.2 |
| | | Exon 4 | F | 0.05˜0.2 |
| | | | R | 0.05˜0.2 |
| | | Exon 7 | F | 0.1˜0.3 |
| | | | R | 0.1˜0.3 |
| | | Exon 8 | F | 0.1˜0.3 |
| | | | R | 0.1˜0.3 |
| | | Exon 9 | F | 0.2˜0.4 |
| | | | R | 0.2˜0.4 |
| | MODY5 | Exon 1 | F | 0.1˜0.3 |
| | | | R | 0.1˜0.3 |
| | MODY6 | Exon 2 | F | 0.1˜0.3 |
| | | | R | 0.1˜0.3 |
| | MODY7 | Exon 2 | F | 0.2˜0.4 |
| | | | R | 0.2˜0.4 |
| A | MODY5 | Exon 2 | F | 0.1˜0.3 |
| | | | R | 0.1˜0.3 |
| | | Exon 3 | F | 0.05˜0.2 |
| | | | R | 0.05˜0.2 |
| | | Exon 4 | F | 0.1˜0.3 |
| | | | R | 0.1˜0.3 |
| | | Exon 7 | F | 0.05˜0.2 |
| | | | R | 0.05˜0.2 |
| | MODY4 | Exon 1 | F | 0.4˜0.6 |
| | | | R | 0.4˜0.6 |
| | MODY4 | Exon 2 | F | 0.5˜0.8 |
| | | R | 0.5˜0.8 | |
| MODY1 | Exon 10 | F | 0.1˜0.3 | |
| | | R | 0.1˜0.3 | |

The composition of a reaction solution used in the multiplex PCR was as follows:

Groups A and B

| | |
|---|---|
| Sterilized DNase- and RNase-free water | 20.4 μl |
| dNTP mix (each nucleotide 2.5 mM) | 5 μl |
| 10 × Taq polymerase buffer | 5 μl |
| a set of primers (14 or 18 primers, 5-30 pmol each) | 14 μl |
| genomic DNA (200-1.0 μg) | 1 μl |
| Taq polymerase (5 unit/μl) | 0.6 μl |

The products of the multiplex PCR conducted in the optimized conditions were analyzed using a lab chip (Agilant Co., U.S.A.).

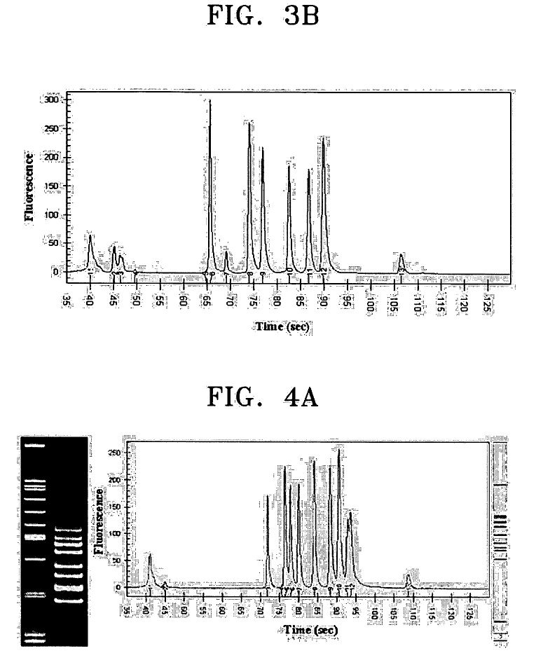

The results of the analysis using the lab chip are shown in FIGS. 4A and 4B. As shown in FIGS. 4A and 4B, all the exons can be amplified above a particular concentration due to the primer concentration optimization. In FIGS. 4A and 4B, peaks indicated by arrows correspond to exons that have been amplified due to the optimization. From the results of FIGS. 4A and 4B, to obtain PCR products with above a predetermined concentration, optimum concentration of each primer within bothh groups A and B may preferably range from 5-30 pmol (0.1-0.6 μM).

### EXAMPLE 5

### Amplification of 9 Exons and 7 Exons of Human MODY Genes 1, 4, 5, 6 and 7 by Multiplex PCR Using Variant Primers

Variant primers were designed by deleting three nucleosides from 3′-terminal of each of primers selected from groups A and B used in Example 4 and adding three nucleosides to 5′-terminal of each of the primers. The designed variant primers are shown in Table 3 below.

TABLE 3
Variant Primers

| Exon | Name of Primer | SEQ ID NO. |
|---|---|---|
| MODY 1 | M1e2fpa | 33 |
| Exon 2 | M1e2rpa | 34 |
| MODY 1 | M1e4fpa | 35 |
| Exon 4 | M1e4rpa | 36 |
| MODY 1 | M1e9fpa | 37 |
| Exon 9 | M1e9rpa | 38 |
| MODY 5 | M5e2fpa | 39 |
| Exon 2 | M5e2rpa | 40 |

PCR was performed using some of the variant primers in Table 3 in the same conditions as in Example 4. The composition of a reaction solution used was as follows.

| | |
|---|---|
| Sterilized DNase- and RNase-free water | 20.4 μl |
| dNTP mix (each nucleotide 2.5 mM) | 5 μl |
| 10 × Taq polymerase buffer | 5 μl |
| a set of primers (14 or 18 primers, 5-30 pmol each) | 18 μl |
| genomic DNA (200-1.0 μg) | 1 μl |
| Taq polymerase (5 unit/μl) | 0.6 μl |

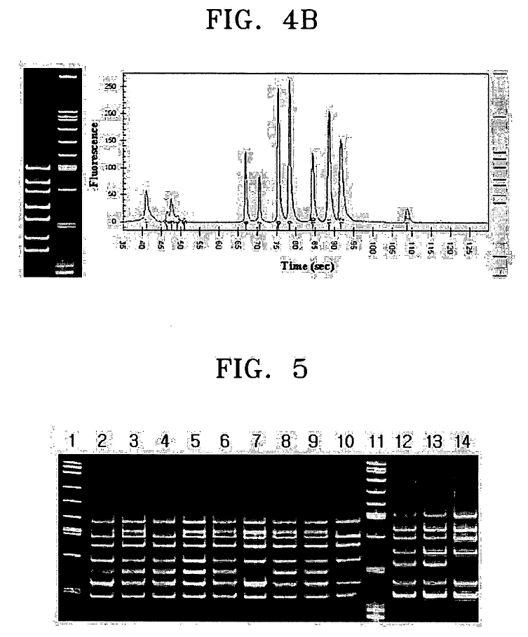

The PCR products were identified by electrophoresis on 6% polyacrylamide gel. Lanes 1 through 14 in FIG. 5 are described in Table 4 below.

TABLE 4

| Lane No. | Description |
|---|---|
| 1 | Molecular weight markers |
| 2 | Products of multiplex PCR using group A |
| | (using variant primers, instead of sets of normal primers, |
| | for exon 2 of MODY1) |
| 3 | Products of multiplex PCR using group A |
| | (containing sets of normal primers for exon 2 of MODY 1) |
| 4 | Products of multiplex PCR using group A |
| | (sets of normal primers for exon 2 of MODY 1 is excluded) |
| 5 | Products of multiplex PCR using group A |
| | (using variant primers, instead of sets of normal primers, |
| | for exon 9 of MODY 1) |
| 6 | Products of multiplex PCR using group A |
| | (containing sets of normal primers for exon 9 of MODY 1) |
| 7 | Products of multiplex PCR using group A |
| | (sets of normal primers for exon 9 of MODY 1 are excluded) |
| 8 | Products of multiplex PCR using group A |
| | (using variant primers, instead of normal primers, for |
| | exons 2 and 9 of MODY 1) |
| 9 | Products of multiplex PCR using group A |
| | (using sets of primers for exons 2 and 9 of MODY 1) |
| 10 | Products of multiplex PCR using group A |
| | (sets of primers for exons 2 and 9 of MODY 1 are excluded) |
| 11 | Molecular weight markers |
| 12 | Products of multiplex PCR using group B |
| | (using variant primers, instead of sets of normal primers, |
| | for exon 2 of MODY 5) |
| 13 | Products of multiplex PCR using group B |
| | (using sets of primers for exon 2 of MODY 5) |
| 14 | Products of multiplex PCR using group B |
| | (sets of normal primers for exon 2 of MODY 1 are |
| | excluded) |

As described in Example 4, group A refers to a primer pool including 9 sets of primers for exons 2, 3, 4, 7, and 9 of MODY 1, exon 2 of MODY 6, and exon 5 of MODY 7. Group B refers to a primer pool including 7 sets of primers for exon 1 of MODY 1, exons 1 and 2 of MODY 4, and exons 2, 3, 4, and 7 of MODY 5.

As is apparent from FIG. 5, all the corresponding exons can be amplified by multiplex PCR using the four sets of variant primers in Table 3.

### EXAMPLE 6

### Identification of Multiplex PCR Products Using Nucleotide Sequencing Analsyis

The multiplex PCR products obtained in Example 4 using the two primer pools, one including the 9 sets of primers and the other including the 7 sets of primers, were purified using a PCR kit. The resulting purified DNAs were sequenced using an ABI3700 and analyzed using software DNAstar™ for comparison with consensus sequences for human MODY genes 1, 4, 5, 6, or 7. As a result, the sequences of the amplified exons almost matched the corresponding consensus sequences, particularly, with 99% homology for exon 1 of MODY 4 and 98-100% homology for the other exons.

As described above, using a multiplex PCR primer pool according to the present invention, exons of human MODY gene 1, 4, 5, 6, or 7 can be amplified in a single reaction tube. The multiplex PCR primer pool according to the present invention can be effectively used in the sequence analysis of exons of human MODY gene 1, 4, 5, 6, or 7. In addition, a target sequence of human MODY gene 1, 4, 5, 6, or 7 can be effectively amplified using a kit according to the present invention.

### Example 7

### Multiplex PCR Amplification of Two Exons of MODY4 Gene Using Additional Multiplex PCR Primers

We have designed additional two multiplex PCR primers for the amplification of MODY4 exons and performed a single PCR and multiplex PCR using the primers. The additionally designed multiplex primers for the MODY4 are shown below in Table 5.

TABLE 5
multiplex primers for the MODY4

| Name | | | Name of | | Size of the PCR |
|---|---|---|---|---|---|
| of gene | Exon No. | Direction | Primer | Sequence | products (bp) |
| MODY4 | Exon 1 | F | M4e1r2n | SEQ ID NO. 41 | 468 |
| | | R | M4e1r2n | SEQ ID NO. 16 | |
| | Exon2 | F | M4e2f1 | SEQ ID NO. 42 | 391 |
| | | R | M4e2r6n | SEQ ID NO. 18 | |

The PCR was achieved by initial denaturation (5 min at 95° C.), 30 cycles of denaturartion (30 sec at 95° C.), annealing (15 sec at 64° C.) and polymerization (30 sec at 72° C.), and final extension (3 min at 72° C.). The multiplex PCR was performed in a single reaction tube containing the primer pool. The compositions of reaction solutions used in the PCR were as follows:

| | |
|---|---|
| Sterilized DNase- and RNase-free water | 14.45 μl |
| dNTP mix (each nucleotide 20 mM) | 0.25 μl |
| 10 × Taq polymerase buffer | 2.5 μl |
| a set of primers (14 primers, 10 pmol each) | 4 μl |
| genomic DNA (200-1.0 μg) | 1 μl |
| Taq polymerase (5 unit/μl) | 0.3 μl |

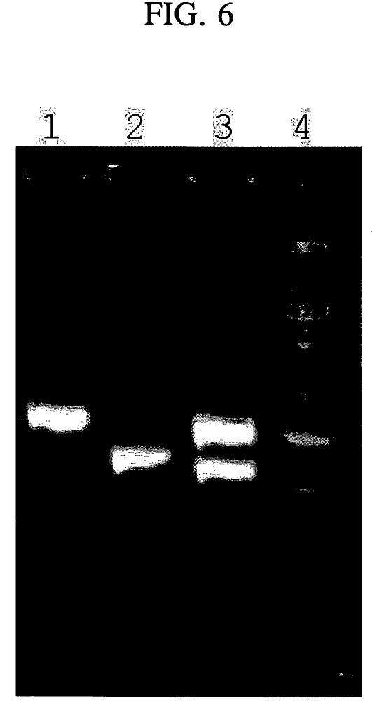

The multiplex PCR products were identified by electrophoresis on 3.5% agarose gel (FIG. 6). As shown in FIG. 6, 2 exons (having expected 461, 391 bp) of human MODY genes 4 could be amplified by single and multiplex PCR using the primer pools. In FIG. 6, lane 1 represents a single PCR product of exon 1 of MODY4, lane 2 represents a single PCR product of exon 2 of MODY4, and lane 3 represents PCR products obtained by multiplex PCR using the primer pool shown Table 5. These multiplex PCR products were purified using a PCR kit. The resulting purified DNAs were sequenced using an ABI3700 and analyzed using software DNAstar for comparison with the corresponding consensus sequences for human MODY genes 4. As a result, the sequences of the amplified exons perfectly matched the corresponding consensus sequences, particularly.

Further, the two primer sets for the MODY4 exons 1 and 2 were used together with other multiplex primers shown in Table 1 in a multiplex PCR and each of the expected PCR product could be obtained by single and multiplex PCR using the primer pools (data not shown).

According to the multiplex PCR primer pools of the present invention, each of the exon of the MODY 1, 4, 5, 6 and 7 genes could be effectively amplified in a single reaction tube.

The multiplex PCR primer pools of the present invention could be very useful for analysing the sequence of the exon of the MODY 1, 4, 5, 6 and 7 genes.

Further, the kit for the amplification of the exon sequences of the MODY 1, 4, 5, 6 and 7 genes could be very useful for the amplification of the exon sequences of the MODY 1, 4, 5, 6 and 7 genes.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A primer pool including at least two sets of primers for amplifying at least two target sequences of human MODY gene 1, 4, 5, 6, or 7, the at least two sets of primers being selected from the group consisting of:
(a) a set of primers including an oligonucleotide having SEQ ID NO. 1 or its variant oligonucleotide and an oligonucloetide having SEQ ID NO. 2 or its variant oligonucleotide;
(b) a set of primers including an oligonucleotide having SEQ ID NO. 3 or its variant oligonucleotide and an oligonucloetide having SEQ ID NO. 4 or its variant oligonucleotide;
(c) a set of primers including an oligonucleotide having SEQ ID NO. 5 or its variant oligonucleotide and an oligonucloetide having SEQ ID NO. 6 or its variant oligonucleotide;
(d) a set of primers including an oligonucleotide having SEQ ID NO. 7 or its variant oligonucleotide and an oligonucloetide having SEQ ID NO. 8 or its variant oligonucleotide;
(e) a set of primers including an oligonucleotide having SEQ ID NO. 9 or its variant oligonucleotide and an oligonucloetide having SEQ ID NO. 10 or its variant oligonucleotide;
(f) a set of primers including an oligonucleotide having SEQ ID NO. 11 or its variant oligonucleotide and an oligonucloetide having SEQ ID NO. 12 or its variant oligonucleotide;
(g) a set of primers including an oligonucleotide having SEQ ID NO. 13 or its variant oligonucleotide and an oligonucloetide having SEQ ID NO. 14 or its variant oligonucleotide;
(h) a set of primers including an oligonucleotide having SEQ ID NO. 15 or its variant oligonucleotide and an oligonucloetide having SEQ ID NO. 16 or its variant oligonucleotide;
(i) a set of primers including an oligonucleotide having SEQ ID NO. 17 or its variant oligonucleotide and an oligonucloetide having SEQ ID NO. 18 or its variant oligonucleotide;
(j) a set of primers including an oligonucleotide having SEQ ID NO. 19 or its variant oligonucleotide and an oligonucloetide having SEQ ID NO. 20 or its variant oligonucleotide;
(k) a set of primers including an oligonucleotide having SEQ ID NO. 21 or its variant oligonucleotide and an oligonucloetide having SEQ ID NO. 22 or its variant oligonucleotide;
(l) a set of primers including an oligonucleotide having SEQ ID NO. 23 or its variant oligonucleotide and an oligonucloetide having SEQ ID NO. 24 or its variant oligonucleotide;
(m) a set of primers including an oligonucleotide having SEQ ID NO. 25 or its variant oligonucleotide and an oligonucloetide having SEQ ID NO. 26 or its variant oligonucleotide;
(n) a set of primers including an oligonucleotide having SEQ ID NO. 27 or its variant oligonucleotide and an oligonucloetide having SEQ ID NO. 28 or its variant oligonucleotide;
(o) a set of primers including an oligonucleotide having SEQ ID NO. 29 or its variant oligonucleotide and an oligonucloetide having SEQ ID NO. 30 or its variant oligonucleotide; and
(p) a set of primers including an oligonucleotide having SEQ ID NO. 31 or its variant oligonucleotide and an oligonucloetide having SEQ ID NO. 32 or its variant oligonucleotide;
(q) a set of primers including an oligonucleotide having SEQ ID NO. 41 or its variant oligonucleotide and an oligonucloetide having SEQ ID NO. 16 or its variant oligonucleotide;
(r) a set of primers including an oligonucleotide having SEQ ID NO. 42 or its variant oligonucleotide and an oligonucloetide having SEQ ID NO. 18 or its variant oligonucleotide,
wherein said variant oligonucleotide is an oligonucleotide having 1 to 3 additional nucleotides joined or deleted from the 3′ end, the 5′ end, or both the 3′ end and the 5′ end of the corresponding oligonucleotide.

2. A method of amplifying at least two target sequences of human MODY gene 1, 4, 5, 6, or 7 comprising performing a polymerization chain reaction using at least two sets of primers selected from the group consisting of:
(a) a set of primers including an oligonucleotide having SEQ ID NO. 1 or its variant oligonucleotide and an oligonucloetide having SEQ ID NO. 2 or its variant oligonucleotide;
(b) a set of primers including an oligonucleotide having SEQ ID NO. 3 or its variant oligonucleotide and an oligonucloetide having SEQ ID NO. 4 or its variant oligonucleotide;
(c) a set of primers including an oligonucleotide having SEQ ID NO. 5 or its variant oligonucleotide and an oligonucloetide having SEQ ID NO. 6 or its variant oligonucleotide;
(d) a set of primers including an oligonucleotide having SEQ ID NO. 7 or its variant oligonucleotide and an oligonucloetide having SEQ ID NO. 8 or its variant oligonucleotide;
(e) a set of primers including an oligonucleotide having SEQ ID NO. 9 or its variant oligonucleotide and an oligonucloetide having SEQ ID NO. 10 or its variant oligonucleotide;
(f) a set of primers including an oligonucleotide having SEQ ID NO. 11 or its variant oligonucleotide and an oligonucloetide having SEQ ID NO. 12 or its variant oligonucleotide;
(g) a set of primers including an oligonucleotide having SEQ ID NO. 13 or its variant oligonucleotide and an oligonucloetide having SEQ ID NO. 14 or its variant oligonucleotide;
(h) a set of primers including an oligonucleotide having SEQ ID NO. 15 or its variant oligonucleotide and an oligonucloetide having SEQ ID NO. 16 or its variant oligonucleotide;
(i) a set of primers including an oligonucleotide having SEQ ID NO. 17 or its variant oligonucleotide and an oligonucloetide having SEQ ID NO. 18 or its variant oligonucleotide;
(j) a set of primers including an oligonucleotide having SEQ ID NO. 19 or its variant oligonucleotide and an oligonucloetide having SEQ ID NO. 20 or its variant oligonucleotide;
(k) a set of primers including an oligonucleotide having SEQ ID NO. 21 or its variant oligonucleotide and an oligonucloetide having SEQ ID NO. 22 or its variant oligonucleotide;
(l) a set of primers including an oligonucleotide having SEQ ID NO. 23 or its variant oligonucleotide and an oligonucloetide having SEQ ID NO. 24 or its variant oligonucleotide;
(m) a set of primers including an oligonucleotide having SEQ ID NO. 25 or its variant oligonucleotide and an oligonucloetide having SEQ ID NO. 26 or its variant oligonucleotide;
(n) a set of primers including an oligonucleotide having SEQ ID NO. 27 or its variant oligonucleotide and an oligonucloetide having SEQ ID NO. 28 or its variant oligonucleotide;
(o) a set of primers including an oligonucleotide having SEQ ID NO. 29 or its variant oligonucleotide and an oligonucloetide having SEQ ID NO. 30 or its variant oligonucleotide; and
(p) a set of primers including an oligonucleotide having SEQ ID NO. 31 or its variant oligonucleotide and an oligonucloetide having SEQ ID NO. 32 or its variant oligonucleotide;
(q) a set of primers including an oligonucleotide having SEQ ID NO. 41 or its variant oligonucleotide and an oligonucloetide having SEQ ID NO. 16 or its variant oligonucleotide;
(r) a set of primers including an oligonucleotide having SEQ ID NO. 42 or its variant oligonucleotide and an oligonucloetide having SEQ ID NO. 18 or its variant oligonucleotide,
wherein said variant oligonucleotide is an oligonucleotide having 1 to 3 additional nucleotides joined or deleted from the 3′ end, the 5′ end, or both the 3′ end and the 5′ end of the corresponding oligonucleotide.

3. The method of claim 2, wherein the polymerization chain reaction is performed using 0.1-1 μM of each of the primers and 100 ng-1 μg of a template DNA.

4. The method of claim 3, wherein the set of primers for amplifying exon 2 of MODY 1 is used at a concentration of 0.1-0.3% M; the set of primers for amplifying exon 2 of MODY 1 is used at a concentration of 0.05-0.2 μM; the set of primers for amplifying exon 4 of MODY 1 is used at a concentration of 0.05-0.2 μM;
the set of primers for amplifying exon 7 of MODY 1 is used at a concentration of 0.1-0.3 μM; the set of primers for amplifying exon 8 of MODY 1 is used at a concentration of 0.1-0.3 μM; the set of primers for amplifying exon 9 of MODY 1 is used at a concentration of 0.2-0.4 μM; the set of primers for amplifying exon 10 of MODY 1 is used at a concentration of 0.1-0.3 μM; the set of primers for amplifying exon 1 of MODY 4 is used at a concentration of 0.4-0.6 μM; the set of primers for amplifying exon 2 of MODY 4 is used at a concentration of 0.5-0.8 μM; the set of primers for amplifying exon 1 of MODY 5 is used at a concentration of 0.1-0.3 μM;
the set of primers for amplifying exon 2 of MODY 5 is used at a concentration of 0.1-0.3 μM; the set of primers for amplifying exon 3 of MODY 5 is used at a concentration of 0.05-0.2 μM; the set of primers for amplifying exon 4 of MODY 5 is used at a concentration of 0.1-0.3 μM; the set of primers for amplifying exon 7 of MODY 5 is used at a concentration of 0.05-0.2 μM; the set of primers for amplifying exon 2 of MODY 6 is used at a concentration of 0.1-0.3 μM; and the set of primers for amplifying exon 2 of MODY 7 is used at a concentration of 0.2-0.4 μM.

5. The method of claim 2, wherein the PCR is achieved by initial denaturation at 90-98° C. for 1-5 minutes, 30 cycles of denaturation at 90-98° C. for 10 seconds to 1 minute, annealing at 60-65° C. for 5 seconds to 3 minutes and polymerization at 70-75° C. for 5 seconds to 5 minutes, and final extension at 70-75° C. for 1-10 minutes.

6. A method of analyzing at least two target nucleotides of human MODY gene 1, 4, 5, 6, or 7 using at least two sets of primers selected from the group consisting of:
(a) a set of primers including an oligonucleotide having SEQ ID NO. 1 or its variant oligonucleotide and an oligonucloetide having SEQ ID NO. 2 or its variant oligonucleotide;
(b) a set of primers including an oligonucleotide having SEQ ID NO. 3 or its variant oligonucleotide and an oligonucloetide having SEQ ID NO. 4 or its variant oligonucleotide;
(c) a set of primers including an oligonucleotide having SEQ ID NO. 5 or its variant oligonucleotide and an oligonucloetide having SEQ ID NO. 6 or its variant oligonucleotide;
(d) a set of primers including an oligonucleotide having SEQ ID NO. 7 or its variant oligonucleotide and an oligonucloetide having SEQ ID NO. 8 or its variant oligonucleotide;
(e) a set of primers including an oligonucleotide having SEQ ID NO. 9 or its variant oligonucleotide and an oligonucloetide having SEQ ID NO. 10 or its variant oligonucleotide;
(f) a set of primers including an oligonucleotide having SEQ ID NO. 11 or its variant oligonucleotide and an oligonucloetide having SEQ ID NO. 12 or its variant oligonucleotide;
(g) a set of primers including an oligonucleotide having SEQ ID NO. 13 or its variant oligonucleotide and an oligonucloetide having SEQ ID NO. 14 or its variant oligonucleotide;
(h) a set of primers including an oligonucleotide having SEQ ID NO. 15 or its variant oligonucleotide and an oligonucloetide having SEQ ID NO. 16 or its variant oligonucleotide;
(i) a set of primers including an oligonucleotide having SEQ ID NO. 17 or its variant oligonucleotide and an oligonucloetide having SEQ ID NO. 18 or its variant oligonucleotide;
(j) a set of primers including an oligonucleotide having SEQ ID NO. 19 or its variant oligonucleotide and an oligonucloetide having SEQ ID NO. 20 or its variant oligonucleotide;
(k) a set of primers including an oligonucleotide having SEQ ID NO. 21 or its variant oligonucleotide and an oligonucloetide having SEQ ID NO. 22 or its variant oligonucleotide;
(l) a set of primers including an oligonucleotide having SEQ ID NO. 23 or its variant oligonucleotide and an oligonucloetide having SEQ ID NO. 24 or its variant oligonucleotide;
(m) a set of primers including an oligonucleotide having SEQ ID NO. 25 or its variant oligonucleotide and an oligonucloetide having SEQ ID NO. 26 or its variant oligonucleotide;
(n) a set of primers including an oligonucleotide having SEQ ID NO. 27 or its variant oligonucleotide and an oligonucloetide having SEQ ID NO. 28 or its variant oligonucleotide;
(o) a set of primers including an oligonucleotide having SEQ ID NO. 29 or its variant oligonucleotide and an oligonucloetide having SEQ ID NO. 30 or its variant oligonucleotide; and
(p) a set of primers including an oligonucleotide having SEQ ID NO. 31 or its variant oligonucleotide and an oligonucloetide having SEQ ID NO. 32 or its variant oligonucleotide;
(q) a set of primers including an oligonucleotide having SEQ ID NO. 41 or its variant oligonucleotide and an oligonucloetide having SEQ ID NO. 16 or its variant oligonucleotide;
(r) a set of primers including an oligonucleotide having SEQ ID NO. 42 or its variant oligonucleotide and an oligonucloetide having SEQ ID NO. 18 or its variant oligonucleotide,
wherein said variant oligonucleotide is an oligonucleotide having 1 to 3 additional nucleotides joined or deleted from the 3′ end, the 5′ end, or both the 3′ end and the 5′ end of the corresponding oligonucleotide.

7. A kit for amplifying a target sequence of human MODY gene 1, 4, 5, 6, or 7 and comprising the primer pool of claim 1.


SEQUENCE LISTING (42 sequences)

SEQ ID NO: 1 - DNA, 21 bp, Artificial Sequence
  primer
ccggctcccaccccagaaggt

SEQ ID NO: 2 - DNA, 24 bp, Artificial Sequence
  primer
ccatgagcccaagtgtgcccattt

SEQ ID NO: 3 - DNA, 26 bp, Artificial Sequence
  primer
cccgggatgaagagatgagagcactg

SEQ ID NO: 4 - DNA, 24 bp, Artificial Sequence
  primer
ggggcctgccactgagtcataaag

SEQ ID NO: 5 - DNA, 23 bp, Artificial Sequence
  primer
agacacccccaccccctactcca

SEQ ID NO: 6 - DNA, 24 bp, Artificial Sequence
  primer
gctgcaaactgggccatgtgaaac

SEQ ID NO: 7 - DNA, 23 bp, Artificial Sequence
  primer
ccctgcaggtcctcctcccacag

SEQ ID NO: 8 - DNA, 23 bp, Artificial Sequence
  primer
actgcgcccggccatattgtctc

SEQ ID NO: 9 - DNA, 25 bp, Artificial Sequence
  primer
atgctggcgtaccctggttgttgag

SEQ ID NO: 10 - DNA, 22 bp, Artificial Sequence
  primer
caaagcggcacagtggggaagc

SEQ ID NO: 11 - DNA, 22 bp, Artificial Sequence
  primer
ggcgtcccaaggcctgaggtct

SEQ ID NO: 12 - DNA, 25 bp, Artificial Sequence
  primer
caatcttgccctttattccctaccc

SEQ ID NO: 13 - DNA, 21 bp, Artificial Sequence
  primer
ggcagggtgggaggggagaac

SEQ ID NO: 14 - DNA, 22 bp, Artificial Sequence
  primer
gcgtcagggtgcagtgggatgt

SEQ ID NO: 15 - DNA, 21 bp, Artificial Sequence
  primer
gcagccatgaacggcgaggag

SEQ ID NO: 16 - DNA, 23 bp, Artificial Sequence
  primer
aggtaaggcggccgggtgagaac

SEQ ID NO: 17 - DNA, 25 bp, Artificial Sequence
  primer
catgttgaacttgaccgagagacac

SEQ ID NO: 18 - DNA, 22 bp, Artificial Sequence
  primer
tgtggcgacgcgctaaggccag

SEQ ID NO: 19 - DNA, 25 bp, Artificial Sequence
  primer
ctcacgtcgctccagcaagaactcc

SEQ ID NO: 20 - DNA, 22 bp, Artificial Sequence
  primer
agtgtggtcgggcgcagtgtca

SEQ ID NO: 21 - DNA, 24 bp, Artificial Sequence
  primer
cagcaacacaacatcccccagagg

SEQ ID NO: 22 - DNA, 25 bp, Artificial Sequence
  primer
tacagcaaccaccaaggccaaatct

SEQ ID NO: 23 - DNA, 21 bp, Artificial Sequence
  primer
gggcctgggcagtccgatgat

SEQ ID NO: 24 - DNA, 25 bp, Artificial Sequence
  primer
aggggagggttcctgggtctgtgta

SEQ ID NO: 25 - DNA, 25 bp, Artificial Sequence
  primer
agaatgtttgcagcgaggggtgtcc

SEQ ID NO: 26 - DNA, 25 bp, Artificial Sequence
  primer
agcggccctaggatcatctcaaaag

SEQ ID NO: 27 - DNA, 25 bp, Artificial Sequence
  primer
atgcccatctccaacccacaactca

SEQ ID NO: 28 - DNA, 25 bp, Artificial Sequence
  primer
gcagggaaaagtgaccaagccaata

SEQ ID NO: 29 - DNA, 24 bp, Artificial Sequence
  primer
gaacgcagaggaggactcactgag

SEQ ID NO: 30 - DNA, 22 bp, Artificial Sequence
  primer
gctgtccatggtaccgtaaggc

SEQ ID NO: 31 - DNA, 23 bp, Artificial Sequence
  primer
cgacggtggcttacaggctaacc

SEQ ID NO: 32 - DNA, 25 bp, Artificial Sequence
  primer
ttccctgcatttgtctcccttctct

SEQ ID NO: 33 - DNA, 21 bp, Artificial Sequence
  primer
aggccggctcccaccccagaa

SEQ ID NO: 34 - DNA, 24 bp, Artificial Sequence
  primer
ccatgagcccaagtgtgcccattt

SEQ ID NO: 35 - DNA, 20 bp, Artificial Sequence
  primer
agacacccccaccccctact

SEQ ID NO: 36 - DNA, 21 bp, Artificial Sequence
  primer
gctgcaaactgggccatgtga

SEQ ID NO: 37 - DNA, 19 bp, Artificial Sequence
  primer
gatggcgtcccaaggcctg

SEQ ID NO: 38 - DNA, 22 bp, Artificial Sequence
  primer
tcttgccctttattccctaccc

SEQ ID NO: 39 - DNA, 21 bp, Artificial Sequence
  primer
atgcagcaacacaacatcccc

SEQ ID NO: 40 - DNA, 22 bp, Artificial Sequence
  primer
agcaaccaccaaggccaaatct

SEQ ID NO: 41 - DNA, 20 bp, Artificial Sequence
  primer
catgaacggcgaggagcagt

SEQ ID NO: 42 - DNA, 20 bp, Artificial Sequence
  primer
ctgcggagccggaggagaac